(19) United States
(12) Patent Application Publication
(10) Pub. No.: US 2005/0244838 A1
(43) Pub. Date: Nov. 3, 2005

(54) Minimizing the meniscus effect

(75) Inventor: Janusz B. Wojtowicz, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/834,647

(22) Filed: Apr. 29, 2004

(51) Int. Cl. C12Q 1/68; C12M 1/34

(52) U.S. Cl. 435/6; 435/287.2; 435/288.7; 436/164; 422/82.05

(57) ABSTRACT

The present application relates to apparatuses and methods for wet-detection of hybridization assays.

### DESCRIPTION

1. Field

The present application relates to systems, devices and methods for wet-detection of biological samples.

2. Introduction

In the biological field, reactions on a solid surface can be used for hybridization assays. A known member of a binding pair on the solid surface can hybridize with a target member of the binding pair from the biological sample to form a duplex in the hybridization fluid. A pattern of duplexed binding pairs on the solid surface provides information about the biological sample. The pattern on the solid surface can be detected to map the information relative to the known members of the binding pairs on the solid surface. In certain instances, it is desirable to control effects of the fluid meniscus on the light for excitation and/or detection of the binding pairs on the solid surface or substrate so that information regarding whether a known member has hybridized with a target member can be accurate. The known members of the binding pair form microarrays. In certain instances, the density of the microarray can lead to positioning the known members near the container wall and thereby increasing the effect of the fluid meniscus.

### SUMMARY

According to various embodiments, a system for wet-detection of biological samples, can include a container including at least one container wall providing a liquid level with a meniscus, wherein the container wall is adapted to provide a liquid surface capable of a flatter meniscus height less than a perpendicular meniscus height, a plurality of emission light sources positioned on the container bottom, wherein the emission light sources are distributed on a critical area of the container bottom, and a detector for collecting light from the emission light sources with substantially no meniscus optical effects.

According to various embodiments, a container for wet-detection of biological samples can include at least one container wall providing a liquid level with a meniscus, wherein the container wall is adapted to provide a liquid surface capable of a flatter meniscus height less than a perpendicular meniscus height, and at least one container bottom adapted for positioning a plurality of emission light sources, wherein the emission light sources are distributed on a critical area of the container bottom.

According to various embodiments, a method for wet-detection of biological samples can include providing a flatter meniscus height less than a perpendicular meniscus height, and detecting light from a microarray with substantially no meniscus optical effects.

According to various embodiments, a system for wet-detection of biological samples can include means for containing the biological sample, wherein the means for containing is adapted to provide a flatter meniscus height less than the perpendicular meniscus height.

### BRIEF DESCRIPTION OF THE DRAWINGS

FIG. 1 illustrates a cross-sectional side view showing the comparison of three different container walls and their respective meniscus according to various embodiments of the present teachings;

FIG. 2 illustrates a perspective view of the three different container walls illustrated in FIG. 1; and

FIG. 3 illustrates a cross-sectional view of a system for wet-detection according to various embodiments of the present teachings.

### DESCRIPTION OF VARIOUS EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

The term “container” as used herein refers to a component used to enclose at least one microarray and hold fluid over the microarray during detection or assay. In various embodiments, the container can be constructed of any material including, but not limited to, metals, glass, plastic, and/or composite material that is compatible with microarray detection. The container can be constructed of different materials such that the container bottom is constructed of one material and the container walls can be constructed of a different material. The container bottom and the container walls can be releasably connected or inseparably connected, while providing a seal to prevent the fluid from migrating between the container bottom and the container walls. In certain embodiments, the container wall can be a gasket and the container bottom can be a glass slide sealed together with an adhesive sealant. The gasket can be constructed of materials known in the art. Certain such materials include elastomeric material such as Silicone Rubber, FDA approved Silicone Rubber, EPDM Rubber, Neoprame (CR) Rubber, SBR Rubber, Nitrile (NBR) Rubber, Butyl Rubber, Hypalon (CSM) Rubber, Polyurethane (PU) Rubber, Viton Rubber, and polydimethylsiloxane (Slygard™ elastomer by Dow Corning). In various embodiments, the container can be constructed of harder plastics such as acrylonitrile-butadiene-styrene plastic, polyurethane, polyvinylchloride, polycarbonate, polyethylene, TEFLON™, polystyrene, KALREZ™, or other materials known in the art of consumables manufacturing. In various embodiments, the container can have any cross-sectional shape including, but not limited to, circular, triangular, rectangular, etc.

The term “excitation light source” as used herein refers to a source of irradiance that can provide excitation that results in fluorescent emission. Light sources can include, but are not limited to, white light, halogen lamp, lasers, solid state laser, laser diode, micro-wire laser, diode solid state lasers (DSSL), vertical-cavity surface-emitting lasers (VCSEL), LEDs, phosphor coated LEDs, organic LEDs (OLED), thin-film electroluminescent devices (TFELD), phosphorescent OLEDs (PHOLED), inorganic-organic LEDs, LEDs using quantum dot technology, LED arrays, filament lamps, arc lamps, gas lamps, and fluorescent tubes. Light sources can have high irradiance, such as lasers, or low irradiance, such as LEDs. The different types of LEDs mentioned above can have a medium to high irradiance.

The term “emission light source” as used herein refers to a potential source of light that can emit fluorescent light and/or chemiluminescent light if properly excited. An example of an emission light source is the oligonucleotide spot which can form the known member of a binding pair, where an array of binding sites for such spots makes up a microarray as described herein. Microarrays can have densities of 4 binding sites, spots, and/or features per square millimeter or up to 10⁴ binding sites, spots, and/or features per square millimeter. Binding sites can be positioned on the substrate by pin spotting, ink-jetting, photo-lithography, and other methods known in the art of high density deposition.

The term “detector” as used herein refers to any component, portion thereof, or system of components that can detect light including a charged coupled device (CCD), back-side thin-cooled CCD, front-side illuminated CCD, a CCD array, a photodiode, a photodiode array, a photo-multiplier tube (PMT), a PMT array, complimentary metal-oxide semiconductor (CMOS) sensors, CMOS arrays, a charge-injection device (CID), CID arrays, etc. The detector can be adapted to relay information to a data collection device for storage, correlation, and/or manipulation of data, for example, a computer, or other signal processing system. The detector can be adapted to collect light from fluorescence, chemiluminescence, etc.

The term “wet-detection” as used herein refers to detecting light through a liquid, where the liquid-air interface affects the light path into and/or out of the liquid.

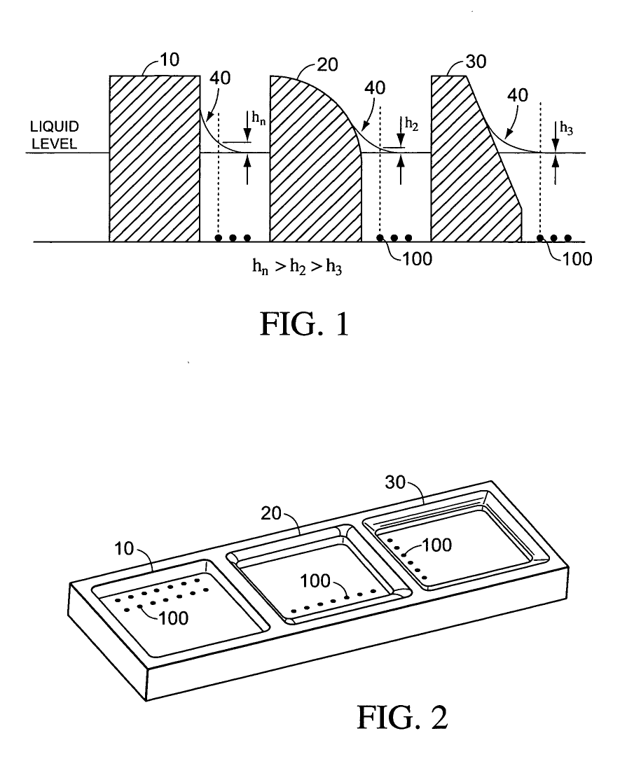

The term “meniscus” as used herein refers to the free surface of a liquid which is near the container walls and which is curved because of surface tension. The term “perpendicular meniscus” refers to the liquid surface near the container wall when the liquid level is perpendicular to the container wall. The term “meniscus height” refers to the height above the liquid level of the meniscus at the boundary of the critical area. An example of this is illustrated in FIG. 1 as the value hₙ or perpendicular meniscus height. The term “flatter meniscus” refers to the liquid surface near the container wall when the meniscus height is less than the perpendicular meniscus height.

The term “critical area” as used herein refers to the portion of the container bottom onto which the microarray is positioned and from which emission light may be emitted. It is desirable that the liquid level over the critical area be substantially flat so as provide substantially no meniscus optical effects. The term “meniscus optical effects” as used herein refers to obscuring and/or shifting of light emitted from emission light sources which can cause problems in gridding and/or quantification.

According to various embodiments, as illustrated in FIGS. 1, different types of container walls provide a different meniscus. FIG. 1 is a comparative diagram showing three walls oriented to face in the same direction to show the different effects on the meniscus. The boundary of the critical area is designated by the vertical broken line where the emission light sources 100 come closest to the container wall. This boundary is substantially the same distance from the container wall as measured where the container wall meets the container bottom. The liquid level is designated by the solid horizontal line labeled as such. Perpendicular container wall 10 provides a meniscus 40 that has a meniscus height, hₙ. Rounded container wall 20 provides a meniscus 40 that has a meniscus height, h₂. Chamfered container wall 30 has a beveled or sloped surface and provides a meniscus 40 that has a meniscus height, h₃. The value of h₂ is less than h₂ and h₂ is less than h₃. It is apparent to one skilled in the art of fluid mechanics that container walls with different shapes can provide differences in contact angle to provide a flatter meniscus with a meniscus height less than the perpendicular meniscus height. According to various embodiments, the contact angle can also be affected by surface energy of the container wall and/or surface tension of the liquid. The surface energy of the container wall can be modified by, for example, coating the container wall or constructing the container wall of different materials. The surface tension of the liquid used in wet-detection can be modified by, for example, changing the composition of the liquid and/or adding surfactant.

According to various embodiments, as illustrated in FIG. 2, the container can include more than one container wall providing a flatter meniscus on all sides of the critical area. For illustrative purposes, FIG. 2 shows a multi-compartment container with three container bottoms enclosed by perpendicular container walls 10, rounded container walls 20, and chamfered container walls 30. According to various embodiments, the container can include one compartment or multiple compartments with one bottom or multiple bottoms with one type of container walls or different types of container walls. For illustrative purposes a few rows of emission light sources 100 are shown in FIG. 2. According to various embodiments, a microarray can contain a series of rows and columns

According to various embodiments, the container can be used for wet-detection of biological samples. The container wall can provide a liquid level with a meniscus such that the liquid surface has a flatter meniscus where the flatter meniscus height is less than the perpendicular meniscus height. The container bottom can provide a surface for positioning the microarray to generate a plurality of emission light sources. The binding pairs of the microarray can be distributed on the critical area of the container bottom. According to various embodiments, the container wall can be rounded. According to various embodiments, the container wall can be chamfered. According to various embodiments, the container wall can be a gasket coupled to a glass slide onto which the microarray is spotted.

According to various embodiments, the container can provide a wet-detection volume less than 300 microliters. Varying amounts of that volume can be filled with liquid for wet-detection up to 300 microliters. The amount of liquid can be 1.0 to 50 microliters.

According to various embodiments, a system for wet-detection of biological samples can include the container with the microarray and a detector for collecting light from the emission light sources of the microarray with substantially no meniscus optical effects. According to various embodiments, the emission light sources can provide fluorescent light. According to various embodiments, an excitation light source or a plurality of excitation light sources can provide excitation light to generate fluorescent light from the emission light sources. According to various embodiments, the emission light sources can provide chemiluminescent light. According to various embodiments, an excitation light source or a plurality of excitation light sources can provide illumination to the container bottom to establish a background to the microarray.

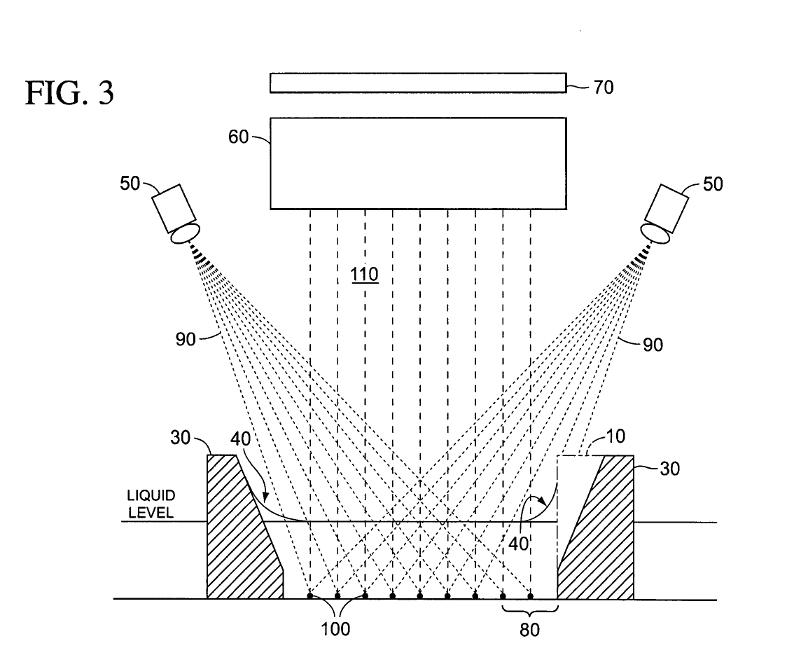

According to various embodiments, as illustrated in FIG. 3, a system for wet-detection can include a container with container walls 30 and container bottom with emission light sources 100. The system can further include excitation light sources 50 which can direct excitation light 90 to emission light sources 100. Emission light sources 100 can emit emission light 110 which can be collected by lens system 60 and captured by detector 70. According to various embodiments, FIG. 3 depicts for illustrative purposes in ghost lines container wall 10 to show another desirable effect of non-perpendicular container walls. This effect in a reduction of shadow 80 due to illumination at an angle by excitation light sources 50. The shadow 80 results from a portion of excitation light 90 being blocked by container wall 10 such that emission light sources 100 in shadow 80 are illuminated by excitation light 90 from only the left excitation light source 50. Shadowing can lead to nonuniform excitation.

According to various embodiments, a method for wet-detection of biological samples can include providing a flatter meniscus height less than the perpendicular meniscus height and detecting light from the microarray with substantially no meniscus optical effects. Portions of the microarray can be activated by binding to form a plurality of potential emission light sources. According to various embodiments, providing excitation light can generate fluorescent light from the emission light sources of the microarray. According to various embodiments, the activation by binding can generate chemiluminescent light from the emission light sources of the microarray. According to various embodiments, detecting can include collecting light emitted or reflected from at least a portion of the microarray for the purpose of recognizing a pattern.

Other various embodiments of the invention will be apparent to those skilled in the art from consideration of the specification and practice of the invention disclosed herein.

1. A system for wet-detection of biological samples, the system comprising:
a container comprising:
at least one container wall providing a liquid level with a meniscus, wherein the container wall is adapted to provide a liquid surface capable of a flatter meniscus height less than a perpendicular meniscus height;
a plurality of emission light sources positioned on the container bottom, wherein the emission light sources are distributed on a critical area of the container bottom; and
a detector for collecting light from the emission light sources with substantially no meniscus optical effects.

2. The system of claim 1, wherein the container wall is rounded.

3. The system of claim 1, wherein the container wall is chamfered.

4. The system of claim 1, wherein the container wall is a gasket.

5. The system of claim 1, wherein the emission light sources provide fluorescent light.

6. The system of claim 5, further comprising a plurality of excitation light sources providing excitation light to the emission light sources.

7. The system of claim 1, wherein the emission light sources provide chemiluminescent light.

8. The system of claim 7, further comprising a plurality of excitation light sources providing illumination to the container bottom.

9. A container for wet-detection of biological samples, the container comprising:
at least one container wall providing a liquid level with a meniscus, wherein the container wall is adapted to provide a liquid surface capable of a flatter meniscus height less than a perpendicular meniscus height; and
at least one container bottom adapted for positioning a plurality of emission light sources, wherein the emission light sources are distributed on a critical area of the container bottom.

10. The container of claim 9, wherein the container wall is rounded.

11. The container of claim 9, wherein the container wall is chamfered.

12. The container of claim 9, wherein the container wall is a gasket.

13. A method for wet-detection of biological samples, the method comprising:
providing a flatter meniscus height less than a perpendicular meniscus height; and
detecting light from a microarray with substantially no meniscus optical effects.

14. The method of claim 13, further comprising providing excitation light to generate fluorescent light from emission light sources on the microarray.

15. The method of claim 13, further comprising generating chemiluminescent light from the emission light sources on the microarray.

16. The method of claim 13, wherein detecting comprises collecting light from at least a portion of the emission light sources.

17. A system for wet-detection of biological samples, the system comprising:
means for containing the biological sample, wherein the means for containing is adapted to provide a flatter meniscus height less than the perpendicular meniscus height.

18. The system of claim 17, further comprising:
means for emitting light from the biological sample.

19. The system of claim 18, further comprising:
means for detecting light from the biological sample.

20. The system of claim 19, further comprising:
means for providing excitation light to the biological sample.

21. The system of claim 20, further comprising:
means for avoiding shadow on the biological sample.
